(19) United States
(12) Patent Application Publication
(10) Pub. No.: US 2008/0221534 A1
(43) Pub. Date: Sep. 11, 2008

(54) Tongue scraper and treatment device

(76) Inventor: Deepty U. Patel, Lutz, FL (US)

Correspondence Address: Office of Donald Grant Kelly, 127 S. FAIRFAX STREET, #140, ALEXANDRIA, VA, 22314, US

(21) Appl. No.: 11/716,003

(22) Filed: Mar. 9, 2007

(51) Int. Cl. A61M 35/00

(52) U.S. Cl. 604/310

(57) ABSTRACT

A tongue treatment device including a handle, a gripping portion and a working end for scraping a tongue and applying a treatment material to the tongue surface. The working end is configured as a closed loop defining an opening. On a first or upper side of an outer portion of the working end is an undulated toothed surface for cleaning and scraping the tongue surface to remove debris and bacteria. A bridge element with an acupressure beaded first surface interconnects opposed lateral sides of the closed loop. The bridge is configured to include at an opposite second surface a reservoir for temporarily holding treatment material. Along the edges of the reservoir are provided rows of massaging teeth for effectively applying the material to the tongue surface. A removable cover is provided to protect against cross contamination and may be positioned over or around the working end while not in use.

### CROSS-REFERENCE TO RELATED APPLICATIONS

The present application is related to applicant's prior application Ser. No. 798,942 filed on Feb. 11, 1997, and now U.S. Pat. No. 5,779,475; the disclosure of which in its entirety is hereby incorporated herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates to device for treatment of tongues as by scraping, stimulating, massaging and applying medication or other substances thereto.

2. Description of the Related Art

The following references are relevant in varied extent to the unique device and method herein described and claimed, but for reasons to be discussed cannot achieve the objectives or desired results of the present invention.

U.S. Pat. No. 790,228 issued to Rohrer illustrates a culinary tool for kitchen use. The tool has a square head with toothed sections designed for removing corn from cobs, shredding pineapple and the like. Patentee Seitz, in U.S. Pat. No. 577,074, depicts a hand tool for forming butter-balls where a working end of the tool includes a curved blade with notches or serrations designed to provide the desired rolling effect while also providing attractive ridges on the butter-ball.

Maxon's fish scaler shown in U.S. Pat. No. 1,519,689 is a hand tool in the form of sheet metal with handle reaches connected to a working portion. At the working end of the working portion is an arcuate array of sharp scale ripping teeth.

In U.S. Pat. No. 4,795,344, Brewer, Jr. discloses a cleaning instrument for removing plaque and calculus deposits from the titanium anchor cylinders of tissue integrated dental prostheses is disclosed. The instrument, molded from hard plastic, such as polypropylene or glass reinforced nylon, has an elongated handle and a substantially crescent-shaped scraper body disposed at one end of the handle. The scraper body defines an interior opening which is of the same internal diameter as the external diameter of the titanium anchor cylinders to be cleaned. Interior walls of the crescent-shaped scraper have teeth or ridges to provide a scraping or cutting action for the calculus deposits.

These devices, Rohrer ('228), Seitz ('074), Maxon ('689) and Brewer, Jr. ('344) clearly are related only in their vaguely similar configurations and their scraping effect, but in no way are conducive to tongue application.

The crescent-shaped body of the Brewer, Jr. instrument extends more than one-half of the corresponding circle. Therefore, in order to place the scraper on the titanium cylinder the jaws of the scraper must be slightly spread apart and thereafter the scraper body snaps onto and is removably held on the titanium cylinders. Back and forth rocking action of the instrument then provides good cleaning action of the cylinders.

While Rohrer ('228), Seitz ('074), Maxon ('689) and Brewer, Jr. ('344), as above discussed, include features of interest in the present context, the devices taught thereby obviously are not designed or configured to function in such a way as to both clean and apply medication to a tongue. However, the field is crowed with a great number of specifically designed tongue scrapers worthy of note. As will be explained they, too, do not enjoy the unique advantages of the present device.

Presently named inventor Patel describes a widely used tongue scraper in the U.S. Pat. No. 5,779,475 noted hereabove. This utility patent illustrates a hand-manipulated tongue scraping instrument set forth in a number of embodiments each of which includes a linear handle with a gripping area terminating in a scraper element.

As will be seen in the Patel ('475) disclosure, the scraper element is formed in a closed loop configuration. A proximate area of the closed loop configuration is integrated into said gripping area and oriented at a slight angle relative to the linear handle to facilitate effective action against the tongue.

An outward or distal portion of the scraper element of Patel ('475) is provided with uniquely shaped tongue scraping or massaging teeth. The teeth on the tongue scraper are configured to define a plurality of triangular cutouts. Such cutouts are in the form of a plurality of triangular wells and teeth members which together define the scraper teeth with a scraping extent. The teeth members include linear beveled side extents and an arcuate top surface. The teeth are formed along less than one quarter of the entire periphery of scraping extent. Triangular wells associated with the teeth have a depth that is less than ⅓ the height of the scraping extent.

While the Patel patented device just discussed is recognized as providing exceptional tongue scraping and massaging effects, it is not configured to temporarily contain and apply topical medication or other materials to the tongue area. This is true of the vast majority of known tongue treatment devices and methods.

U.S. Pat. No. 1,701,616 granted to Gross illustrates a triangular looped tongue scraper with an edged that is beveled apparently to avoid or preclude injury to the tongue. The Gross device addresses the problem of accumulation of matter in the form of a coating. In use, the triangular portion is insertable into the mouth so as to clean the sides of the tongue as well as the center.

Patentee Moore, in U.S. Pat. No. 2,574,654, describes a tongue cleaner detachably mounted on a toothbrush handle. The Moore device includes a scraper blade with a serrated edge as well as a smooth edge. In one embodiment the blade is connected between two arms attached to a handle mounting channel. In a second embodiment, the blade is connected to the handle through a trapezoidal plate, in lieu of the pair of arms.

The U.S. Pat. No. 5,217,475 issued to Kuber features a normally planar tongue scraper. The scraper has free ends and a central extent therebetween. The central extent has a first edge and a second edge with one of the edges having teeth formed therein. The teeth are undulating in configuration with arcuate exterior tips adapted to scrape the tongue of a user.

Kuber's scraper is formed of a flexible material to allow bending along the length during use thereof. In an alternate embodiment, a support device is employed for removably retaining the ends of the scraper with the central extent in an arcuate configuration. The support device has a handle end and forwardly projecting spaced fingers with slots therein of a size to receive the ends of the scraper.

Patentee Castanedo's U.S. Pat. No. 3,890,964 teaches a hygienic tongue cleaning and massage instrument for freeing and removing pathogenic foreign matter entrapped in the papillae of the dorsal mucosa and massaging and scraping the papillae to maintain the papillae in healthy condition. The instrument comprises an elongated handle formed at one end with longitudinally diverging arms.

Divergent ends of the arms of Castanedo's cleaning and massage instrument are interconnected by a longitudinally bowed cross-bar having a laterally centered arched portion. It further includes beveled edge faces defining leading concave contact corners of an angle less than ninety degrees to be alternately engaged in line contact with side marginal edges of the tongue as well as medial depressed area of the tongue. When pressed firmly against the tongue and drawn forwardly from the base of the tongue to the tip, the instrument forces entrapped foreign matter from between tongue papillae and propel it forwardly to the tip to be expelled.

A patent granted to Barkwill, U.S. Pat. No. 1,811,775, shows a tongue scraping device with a handle and a part thereof bent at right angles to the handle. The part includes a curved and beveled edge so that this edge can be drawn over the tongue thus scraping the surface of the tongue. At the junction of the part with the handle, a cup is formed to receive material scraped from the tongue.

U.S. Pat. No. 1,893,524 issued to Shanley illustrates a simple, arcuate hand-operated tongue scraping device. Herrara's U.S. Pat. No. 5,032,082 illustrates a denture adhesive removing head affixed to one end of an elongated handle. The head has several lines of projections extending from a common surface. The outer ends of the projections lie on a convexly curved surface. The head and projections are molded from a material that is pliable when warm and relatively rigid when cooled.

Patentee Schultheiss, in his U.S. Pat. No. 4,780,923, presents an interproximal brush handle including a grip portion and an end portion and a transverse hole for accommodating a stem of an interproximal brush. The brush stem is retained securely in a longitudinal groove on closure of a cap which is pivotally attached to the end portion.

Goodman's double headed working tool for cleaning teeth is described in U.S. Pat. No. 2,677,843. Duncan's U.S. Pat. No. 1,586,031 illustrates a hand wielded massaging device with a row of flexible teeth defining arched spaces therebetween.

Again, the above devices illustrate a variety of tongue scrapers and/or stimulators, but none is readily capable of delivering discrete, controlled doses of medication, soothing gel or other tongue treatment substances while at the same time effectively massaging and stimulating the tongue, so as to work the treatment substances into the tongue's papillae.

There are known devices for both scraping and topical application of treatment substances. For example, Runnels presents, in U.S. Pat. No. 2,583,750, a tongue scraper with an elongated handle and a scraper head which is elliptical in shape. Slanted ridges on the surface of the head define pockets that may hold and distribute mouth wash or other preparation used for removal of tongue coatings and for other purposes. Because of the slanted ridge surfaces, particles removed from the tongue will collect in these pockets during movement of the head. Moreover, the scraper head is closed (rather than an open loop) and does not permit passage of material and tongue coating substances through the head.

Somewhat similar to Runnels ('750), Millner presents in U.S. Pat. No. 5,766,193 a tongue scraper device with scraping pins, wherein the device further features open-ended through-holes arranged to retain liquid medication therein by means of capillary action. Motion across the tongue by Millner's scraper device permits deposit of the liquid from the through-holes.

In both the Runnels and Millner devices, the majority of the head surface is continuous as compared to the open loop featured in the present invention. There is no bridge device serving to both contain a treatment substance and cooperate with arcuate stimulating members to ensure thorough removal of particles from tongue papillae, while substantially at the same time operating to deposit medication.

While a significant number devices will be found in the crowded field of tongue and mouth treatment implements, none has the unique effect or efficiency of combining tongue scraping and stimulation while substantially simultaneously facilitating application of medication to the tongue area. The present invention addresses this long standing need, as will be described in detail herebelow.

### BRIEF SUMMARY OF THE INVENTION

Describing the present invention in general, a significantly improved tongue treatment device is presented, and will be seen as comprising a hand manipulated tool including three major portions, namely a device handle portion and a device working end or treatment portion separated from said handle portion by an intermediate gripping zone. The handle portion includes a free end typical of many hand tools.

The working end of the device comprises a closed loop structure interconnected at a proximal loop portion to said intermediate gripping zone. The closed loop structure further includes an arched distal working loop portion and opposed side loop portions interconnected at said proximal loop portion of said device. The closed loop structure defines an opening therethrough.

The distal working loop portion includes a first array of teeth or ripple elements positioned along its generally arcuate surface for direct application to a tongue. These teeth may be spaced from one another by cut-outs and each may be, for example, generally triangular (or an equivalent shape) so as to provide a vigorous scraping and massaging effect when engaged with the tongue. In this context, of course, the term “cutouts” does not necessarily mean that portions of the teeth or ripple elements are actually formed by cutting. They may be molded or otherwise formed to this configuration. Also, the phrase “vigorous scraping” should not be construed as in any way damaging to the tongue.

Located between said proximate loop portion and the arced distal loop portion of the treatment device is a bridge element spanning and interconnecting the opposed side loop portions. The bridge element has an upper or first surface and a lower or second surface, and may itself have an arched shape substantially similar in form to the arched distal loop portion. Along its first surface, the bridge element may include a plurality of tongue massaging or acupressure beads. These beads collaborate with the first array of scraping/massaging teeth. The lower or second surface of the bridge element displays a configuration considerably different from that of the first surface as will be described herebelow.

Along a lower or second surface of the bridge, namely the surface opposite the first surface just described, is defined a shallow, temporary storage area or reservoir configured as a slot or channel to hold or retain substances of a medical and/or soothing nature. This storage reservoir area can comprise a unit which is integrally formed within the bridge, or a unit which may be either temporarily attached or permanently affixed to the bridge.

In terms of depth, the reservoir channel does not extend entirely through the bridge thickness, but does extend to a depth that is at least sufficient to contain an adequate supply of treatment substance (in liquid, powder or slurry form). The reservoir channel is formed so as to extend from substantially adjacent one side loop portion to another side loop portion. The reservoir channel is open-ended so as to facilitate effective dispensing of treatment substance therefrom. It also helps avoid treatment substance from being bound or trapped within the reservoir. Having the reservoir channel extended along the bridge from side to side of the treatment device effectively assures broad engagement with the tongue. This facilitates thorough application of the treatment substance to a broad portion of the tongue.

Bordering at least one edge of the storage slot on the second side of the bridge may be a first array of massaging teeth elements. These massaging teeth elements may coextend along the entire reservoir, and may be configured of a smoothly undulating surface of interconnected outwardly extended arches (or ripples) defining similarly arched spacing therebetween. This configuration is intended to be less aggressive than the aforementioned scraping teeth. Instead, the smooth undulating surfaces are intended to have a soothing and massaging treatment effect. This undulating configuration is advantageous in gently spreading and thoroughly applying treatment substances, without unduly scraping away the substance just applied.

Further cooperating with these massaging teeth disposed along the bottom or second side of the bridge element is another set of gentle massaging teeth formed at the distal working loop portion, generally opposite to the scraping teeth located at a first or top side of the distal working loop portion so as to be applied directly to the tongue.

A removable cover is provided to prevent cross contamination. Obviously, a tongue treatment device soiled by bacteria or other substances should be covered to prevent external contamination thereby. Then, too, an unused treatment device must be kept clean pending future application. Such a cover may take any of several forms. For example, it may be snapped on or press-fit into place over the application end thereof, as will be described in detail herebelow.

For a better understanding of the invention, its operating advantages and the specific objects attained by its uses, reference should be had to the accompanying drawings and descriptive matter.

### BRIEF DESCRIPTION OF THE SEVERAL VIEWS OF THE DRAWINGS

FIG. 1 is an illustration of the tongue treatment device viewing a first or top side thereof and constructed in accordance with the principles of the present invention;

FIG. 1A illustrates a sectional view of a portion of the treatment device distal loop end portion, wherein the section is taken at section lines 1A-1A on FIG. 1;

FIG. 2 is a plan view of a lower or second surface of the treatment device;

FIG. 3 is a side view of the tongue treatment device;

FIG. 3A is an end view of the tongue treatment device distal loop portion as viewed in direction 3A in FIG. 3;

FIG. 4 is a plan view of the unique tongue treatment device with cover attached;

FIG. 5 is a perspective view of the cover device in closed position;

FIG. 5A is a perspective view of the cover device in open position;

### DETAILED DESCRIPTION OF THE INVENTION

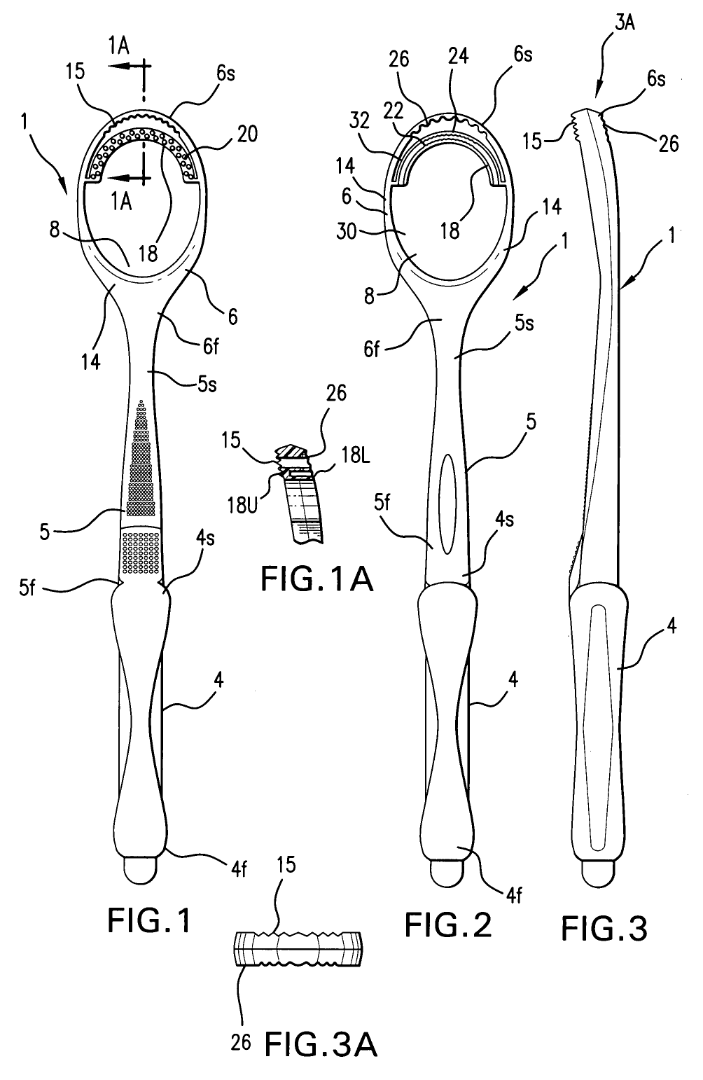

With particular reference to FIGS. 1-3, the improved tongue treatment device 1 is presented as including three principal portions, namely a device handle portion 4, an intermediate gripping zone 5 and a working end 6. Said handle portion has a free end 4f and a second end 4s disposed adjacent the intermediate gripping zone 5. The handle gripping zone includes a first end 5f interconnected at or integral with handle portion second end 4s. The handle gripping zone 5 also includes a second end 5s integral with or interconnected at first end 6f of the device working end or treatment portion 6. All components of this treatment device may be integrally fabricated.

The working end 6 of the treatment device 1 comprises a closed loop structure interconnected at its proximal loop portion 6f to the intermediate gripping zone second end 5s. The closed loop structure further includes a distal working loop portion or second end 6s with an arc configuration, and laterally opposed side loop portions 14. The side loop portions 14 are joined at proximal loop portion 6f. The closed loop structure defines an opening 8 therethrough.

Along the arc of distal working loop portion 6s is disposed a first array of undulated upper or first surface teeth elements 15 for application to a tongue. The undulated first surface teeth elements 15 may be generally triangular in shape and spaced by cut-outs. While the exact shape, triangular or otherwise, is not critical, it is important that these teeth be formed so as to deliver a vigorous yet non-damaging raking and massaging effect while applied in removing bacteria and debris from the tongue.

Turning again to the loop portion of treatment device 1, it is seen in FIGS. 1 and 2 that a bridge element 18 spans opening 8 and serves to interconnect laterally opposed side loop portions 14 and defining open spaces 30 and 32 in the defined opening 8. This bridge element 18 may be formed as an arc which is generally similar in form to, and spaced from, the arc of distal working loop portion 6s. As seen in FIGS. 1, 1A and 2, bridge element 18 includes an upper or first surface 18u and a lower or second surface 18L. Along the upper surface 18u as clearly illustrated in FIG. 1 bridge element 18 includes a plurality of spaced massaging acupressure beads 20. These beads 20 are configured and located so as to collaborate with teeth elements 15 in raking and massaging the tongue.

The bridge element 18 second or lower surface 18L, best viewed in FIG. 2 and FIG. 1A, is configured to define a reservoir channel 22 for temporary retention and application of material in the form of fluid or powder. Such material, hereafter referred to as treatment substance, may have medicinal properties and/or could be a material associated with cleansing, freshening or soothing the tongue. As shown, the channel 22 is open-ended and defined by walls extending into said second or lower bridge surface 18L. Alternatively, the reservoir channel 22 may take the form of a separate element attached to bridge element 18 or may be formed integral therewith as illustrated. In any case, the defined reservoir channel 22 extends across opening 8 substantially along bridge element 18 to adjacent opposed loop portions 14. This extended channel affords a broad range of treatment substance application along a tongue.

Reservoir channel 22 is shown in FIG. 2 as open-ended adjacent the interconnection of bridge element 18 to opposed loop portions 14. Channel 22 is of such depth and volume to deliver an effective dose of treatment substance. As illustrated in FIG. 1A, channel 22 has a depth that extends almost entirely through the thickness of bridge element 18. Extending along at least one edge of channel 22 is an additional row of massaging elements formed as rounded teeth 24 with smoothly undulating surfaces shaped as outwardly extended waves separated by inwardly arched or wave spacing therebetween.

As shown in FIG. 2, rows of massaging teeth 24 border the two edges of reservoir channel 22. So configured, teeth 24 gently spread and apply the treatment substance without scraping away substance just applied. Spreading and application of treatment substance from reservoir 22 is further facilitated by yet another set of gentle massaging teeth 26 disposed along an underside of distal working loop portion 6s, as best viewed in FIGS. 2 and 3. Channel 22 has an arc configuration formed to complement the arc of distal working loop portion second end 6s. This shape encourages egress of treatment substance from channel 22 open ends.

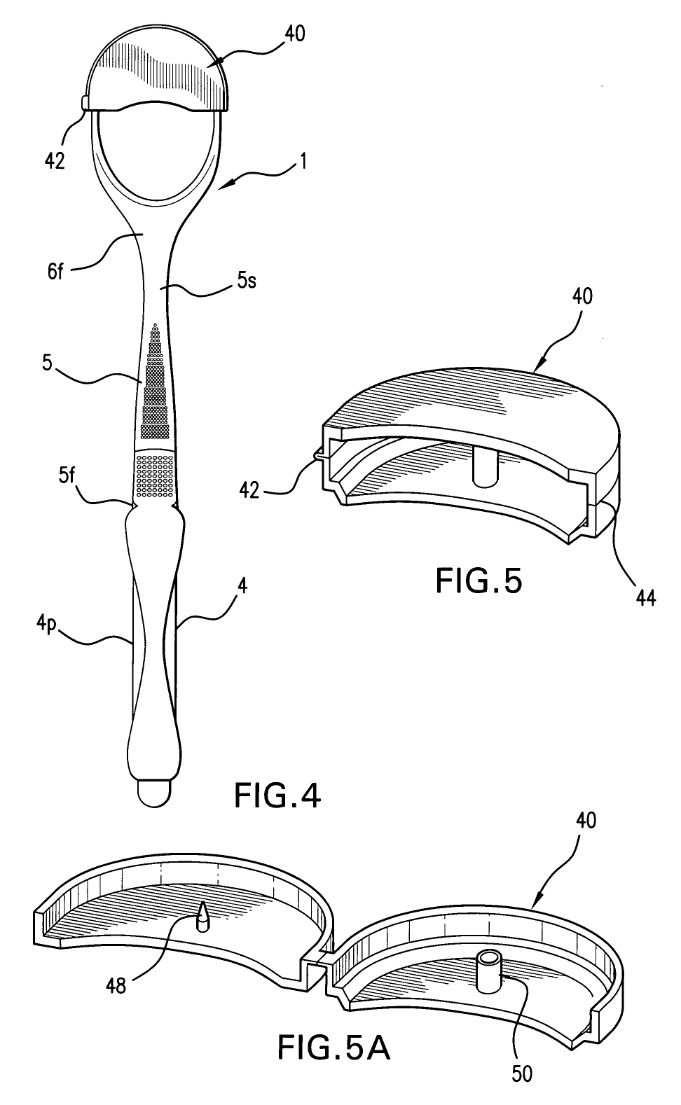

A removable cover 40 is provided as illustrated in FIGS. 4, 5 and 5A and may be snapped onto device 1 or otherwise secured thereto during periods of non-use so as to avoid cross contamination by bacteria and debris or other unwanted sources. The cover 40 may be, as illustrated in FIGS. 5 and 5A, a hinged, interference fit device that fastens together as at 44 when closed. The cover 40 is further shown as including an integral hinge 42, but any suitable hinge would clearly suffice. To help align and secure closure of cover 40, an internal pin and guide hole (48, 50 respectively) are provided within the cover structure and are dimensioned to also have a friction fit relationship therebetween.

The hand manipulated tongue treatment device 1 may be utilized in a manner well known in the art to massage and stimulate the tongue, removing debris therefrom such as food particles, film and bacteria. Following removal of the cover 40, the device 1 is inserted into the mouth and manipulated such that beads 20 on bridge element 18 and undulated upper surface teeth 15 at the distal end 6s of the treatment engage the tongue surface. Movement of treatment device 1 results in a collaboration of beads 20 and teeth 15 along the surface of the tongue.

An undulation effect occurs as portions of the tongue rise into and out of spaces 30 and 32 of opening 8 between the bridge element 18 and loop portion 14. This unique movement serves to vigorously stimulate, exercise and effectively clean, scrape or scrub the tongue of bacteria, food particles, coatings and other foreign matter which may flow out through the opening between bridge element 18 and loop portion 14.

As needed, the second or lower surface 18L of bridge element 18 can be employed to apply treatment substances to tongue areas. This is achieved by charging the storage reservoir channel 22 with a treatment substance (not shown). Charging may be accomplished, for example, by direct application or placement from a supply tube or by dipping the treatment device 1 into a substance container so as to temporarily fill channel 22. Subsequently, the charged channel 22 is brought against the tongue surface.

Manually imparted movement of the bridge element 18, along with the spreading action of teeth 24 and 26 bordering reservoir 22, serves to dispense and disburse the applied substance. Because of the relative movement of the device and tongue, the treatment substance will flow or be drawn from the reservoir channel 22 at the second or lower surface 18L and/or from openings at the ends of reservoir or slot 22. The massaging action ensures effective integration of the treatment substance into the tongue's papillae.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described. Accordingly, all suitable modifications and equivalents may be resorted to, falling within the scope of the invention as defined in the following claims.

What is claimed is:

1. A tongue treatment device comprising a hand manipulated tool including a handle portion interconnected to a working end, said device further characterized by:
said working end comprising a closed loop portion configured to define an opening therethrough and having a first and second working surface;
said closed loop portion includes a loop outer end portion with arc configuration and further includes laterally opposed loop side portions;
said loop outer end portion further includes at said second working surface an array of massaging teeth elements;
said working end further includes a bridge element with a length sufficient to interconnect said opposed side loop portions, said bridge element having a first and second bridge surface;
said bridge element is configured to define therein a shallow reservoir configured to open at said second bridge surface and to temporarily retain therein at least one tongue treatment substance;
whereby said treatment device bridge element cooperates with said massaging teeth on said outer loop end portion to massage the tongue surface while said reservoir deposits said at least one treatment substance thereon.

2. The tongue treatment device of claim 1, wherein:
said first working surface of said loop outer end portion of said treatment device includes an undulated first surface configured to have a vigorous scraping effect when moved along said tongue surface;
whereby said treatment device may alternately be employed to apply a treatment substance to said tongue and to scrape said tongue surface to remove unwanted debris therefrom.

3. The tongue treatment device of claim 2, further characterized by:
said bridge element includes on said first bridge surface an array of bead elements arranged along said bridge to cooperate with said loop outer end portion undulated surface so as to stimulate said tongue.

4. The tongue treatment device of claim 1, further characterized by:
said bridge element includes on said first bridge surface an array of acupressure massaging beads arranged along said bridge to stimulate said tongue.

5. The tongue treatment device of claim 1, further characterized by:
said reservoir is a channel defined by walls extending into said second bridge surface, said slot extending substantially the length of said bridge element;
whereby said slot retains and delivers a supply of treatment substance across said tongue.

6. The tongue treatment device of claim 5, further characterized by:
said channel is defined so as to have at least one end thereof open to permit additional outflow of said treatment substance to the tongue.

7. The tongue treatment device of claim 5, further characterized by:
said reservoir is defined so as to have both ends thereof open to permit outflow of said treatment substance;
said bridge and its defined channel have an arc configuration generally similar in form to the arc configuration of said loop outer end portion;
whereby movement of said bridge relative to a tongue area will facilitate flow of treatment substance from said reservoir ends.

8. The tongue treatment device of claim 4, further characterized by:
said bridge element includes on said second bridge surface at least one row of massaging elements disposed along said reservoir;
whereby said massaging elements may act to disburse said treatment material expressed from said reservoir so as to spread the material along the tongue.

9. The tongue treatment device of claim 4, further characterized by:
said bridge element includes on said second bridge surface a row of massaging teeth disposed along each edge of said reservoir
whereby said massaging teeth may act to disburse said treatment material expressed from said reservoir so as to spread t he material along the tongue.

10. The tongue treatment device of claim 1 wherein said handle portion and said working end are interconnected by an intermediate gripping portion, and all treatment device components are integrally fabricated.

11. The tongue treatment device of claim 1 further including a removable cover configured to fit over the loop outer end portion thereof, covering at least said outer end portion and bridge element so as to protect against cross contamination.

12. The tongue treatment device of claim 11 wherein said removable cover comprises two portions joined by a hinge and further includes a fastener to secure the cover parts in place on said treatment device.

13. The tongue treatment device of claim 12 wherein said cover parts further include:
an internal pin and guide hole defined within said cover to align the parts in closing;
said pin and guide hole are dimensioned to have a friction fit therebetween.
